(19) United States
(12) Patent Application Publication
(10) Pub. No.: US 2023/0212615 A1
(43) Pub. Date: Jul. 6, 2023

(54) GRNA TARGETING CTGF GENE AND USE THEREOF

(71) Applicant: GUANGZHOU REFORGENE MEDICINE CO., LTD., Guangzhou (CN)

(72) Inventors: Junbin Liang, Guangzhou (CN); Hui Xu, Guangzhou (CN); Jiayu Ou, Guangzhou (CN); Bo Gu, Guangzhou (CN)

(21) Appl. No.: 18/188,228

(22) Filed: Mar. 22, 2023

Related U.S. Application Data
(63) Continuation-in-part of application No. PCT/CN2021/095788, filed on May 25, 2021.

(30) Foreign Application Priority Data
Sep. 22, 2020 (CN) 202011002055.8

(51) Int. Cl. C12N 15/90; C12N 15/11; C12N 9/22; C12N 15/88

(52) U.S. Cl. CPC C12N 15/907; C12N 15/11; C12N 9/22; C12N 15/88; C12N 2310/20; C12N 2800/80

(57) ABSTRACT

Provided are gRNA that can direct a Cas enzyme to target a CTGF gene and the use thereof, which belongs to the technical field of gene editing. The gRNA may direct the Cas enzyme to perform targeted cleavage on an SMAD binding site region of a CTGF gene promoter, or the gRNA may direct the Cas enzyme to perform targeted cleavage on a CTGF gene exon 2 region. The gRNA can reduce the overexpression of the human CTGF gene via a CRISPR-Cas gene editing system. The above-mentioned gRNA is used for preparing a drug for use against fibrotic diseases.

### CROSS-REFERENCE TO RELATED APPLICATION

This application is a continuation-in-part of International Application PCT/CN2021/095788, filed May 25, 2021, which claims priority to Application No. CN202011002055.8, filed Sep. 22, 2020, the content of each of which is hereby incorporated by reference in its entirety.

### SEQUENCE LISTING

The instant application contains a Sequence Listing which has been submitted electronically in XML format (ST.26) and is hereby incorporated by reference in its entirety. Said XML copy, created on Mar. 22, 2023, is named P22419796US.SEQ and is 67.4 KB in size.

### TECHNICAL FIELD

The present disclosure relates to the field of gene editing technology, in particular relates to a gRNA targeting CTGF gene and a use thereof.

### BACKGROUND

Idiopathic pulmonary fibrosis (IPF), sometimes called usual interstitial pneumonia (UIP), is a chronic, progressive and fibrotic interstitial lung disease, with a lung histology characteristic of common interstitial pneumonia. IPF is the most common type of idiopathic interstitial pneumonia (IIP). According to the course of the disease, IPF can be divided into cute, subacute, and chronic. The disease is mostly sporadic. According to statistics, the prevalence rate in the overall population is about (2-29)/100,000 per year, with a trend of increase. The average survival after IPF diagnosis is only 2.8 years, and the mortality rate is higher than that of most tumors.

The occurrence of IPF is related to the activation of the TGFβ signaling pathway. Bronchoalveolar lavage fluid (BAL) of patients with IPF contains lots of TGF-β, including its active form TGF-β1. Because TGF-β1can increase connective tissue synthesis, downregulates connective tissue protease, and increases connective tissue protease inhibitors, it is one of the most potent regulators of connective tissue synthesis. TGF-β1 can also induce the production of many growth factors and cytokines involved in fibrosis, including connective tissue growth factor (CTGF), FGF-2, PDGF, insulin-like growth factor, and interleukin.

TGFβ conducts profibrotic signals downstream through connective tissue growth factor (CTGF), and SMAD proteins (Sma and Mad proteins) are important components of this signaling pathway. SMAD proteins play a key role in transmitting TGFβ signals from the cell membrane to the nucleus. After phosphorylated by activated BMPR1 receptors, SMAD proteins (SMAD1, SMAD5 and SMAD8) will detach from cell membrane receptors, and bind to SMAD4 molecules (common SMAD, Co-SMAD) in the cytoplasm, then enter the nucleus. In the nucleus, SMAD multiple complexes act on the CTGF gene with the participation of other DNA-binding proteins to regulate the transcription of the CTGF gene.

In normal cells, the expression of CTGF is very low. In IPF, CTGF is expressed in large quantities in alveolar epithelial cells and fibroblasts, conducting profibrotic signals downstream. For CTGF protein targets, existing antibody drugs have shown good results in clinical trials. According to a document, the GTGTCAAGGGGTC (SEQ ID NO: 57) sequence adjacent to the SMAD binding site is also a response element of TGFβ (J Clin Pathol: Mol Pathol 2001; 54:192-196).

At present, there is still an urgent clinical need for drugs for the treatment of IPF.

Gene editing technology is a technology that aims to specifically change the target sequence of genetic material. In recent years, technologies like zinc finger nuclease (ZFN), transcription activator-like effector nuclease (TALEN), regular clustering of short palindrome repeats (CRISPR) and base editing (BE) have emerged one after another, not only provide powerful tools for gene function research, but also give new treatment options for life medicine.

### CONTENT OF THE PRESENT APPLICATION

In the first aspect, the present application provided a gRNA targeting CTGF gene, wherein the gRNA targets the SMAD binding site region of CTGF gene promoter, or the gRNA targets CTGF gene exon 2 region.

The present application provided a gRNA, wherein the gRNA can guide a Cas enzyme to target and cleave the SMAD binding site region of a CTGF gene promoter, or the gRNA can guide a Cas enzyme to target and cleave the CTGF gene exon 2 region.

In one embodiment, the gRNA provided by the present application can guide a Cas enzyme to target and cleave the SMAD binding site region of a CTGF gene promoter.

In another embodiment, the gRNA provided by the present application can guide a Cas enzyme to target and cleave CTGF gene exon 2 region.

In one embodiment, the Cas enzyme is CRISPR-related nuclease molecules or fusion proteins thereof, consisting of Type II, V, and VI nucleases, wherein Type II nuclease is Cas9, Type V nuclease is Cas 12 and Type VI nuclease is Cas 13. The Cas9 includes but not limited to SpCas9, SaCas9, Nme2Cas9, Nme3Cas9, CjCas9, NmCas9, FnCas9, nCas9, and dCas9 molecules, and fusion proteins and mutants thereof. The Cas12 includes but not limited to Cas12a, Cas12b, Cas12c, Cas12d, Cas12e, Cas 12f, Cas12g, Cas12h, Cas12i, Cas12j, Cas12k, Cas121 and Cas12m molecules, and fusion proteins and mutants thereof

In one embodiment, the Cas enzyme is Cas9.

In one embodiment, the Cas enzyme is AsCpf1.

It can be understood that the SMAD binding site region comprises an adjacent sequence of a single chain where the SMAD binding site is located, and an adjacent sequence of a single chain where the reverse complementary sequence of the SMAD binding site is located.

The gRNA of the present application can guide a Cas nuclease to target and cleave human CTGF gene to change CTGF gene sequence (such as promoter, exon, etc.), such that the expression of CTGF gene is down-regulated by >2%, >5%, >10%, >15%, >20%, >30%, >40%, >50%, >60%, >70%, >80% or >90%; including but not limited to down-regulate the expression after double-stranded break (DSB) is introduced by the gRNA of the present application and CTGF gene is repaired by NHEJ or HDR pathway, and down-regulate the expression of CTGF though destroying the function of the corresponding sequence by replacing bases using the gRNA of the present application.

In order to obtain the gRNA capable of guiding Cas enzymes to target and cleave CTGF gene, the inventor found that the sequence of human CTGF gene is located at chromosome 6 after research, investigation and experimental verification, and the promoter region of the gene contains a SMAD protein binding site, and the sequence of the site is highly conserved in human and mice. The SMAD binding site of human CTGF gene promoter is located at the upstream of the transcription initiation site and whose sequence is CAGACGGA. Therefore, the SMAD binding site region or exon 2 region of CTGF gene promoter is used as a targeting region for CRISPR gene editing, and experiments proved that CTGF gene promoter had good editing efficiency.

The present application compared gRNA and CRISPR-Cas system targeting CTGF gene promoter with gRNA and CRISPR-Cas system targeting and cleaving other targets (such as TGFβ response elements), experiments showed that targeting the SMAD binding site region of promoter can significantly reduce the overexpression of CTGF gene, and had a better editing effect.

In one embodiment, the SMAD binding site region is shown in SEQ ID NO: 38 or a reverse complementary sequence thereof, and sequence of the CTGF gene exon 2 region is shown in SEQ ID NO: 39 or a reverse complementary sequence thereof

In one embodiment, the SMAD binding site region is shown in SEQ ID NO: 40 or a reverse complementary sequence thereof

It can be understood that the SMAD binding site sequence is CAGACGGA, and the region within 10 nucleotides upstream or downstream of the sequence or its reverse complementary sequence (ie, the region shown in SEQ ID NO: 38) can be targeted to edit CTGF gene. Further, the targeting domain targets a region within 5 nucleotides upstream or downstream of SMAD binding site CAGACGGA or its reverse complementary sequence (ie, the region shown in SEQ ID NO: 40); and further, the targeting domain targets the SMAD binding site CAGACGGA or its reverse complementary sequence.

In one embodiment, the SMAD binding site sequence is shown in SEQ ID NO: 40 or a reverse complementary sequence thereof

In one embodiment, the SMAD binding site sequence of CTGF gene promoter is CAGACGGA or a reverse complementary sequence thereof

In one embodiment, the gRNA comprises a targeting domain, and the targeting domain is selected from:

(1) Any of a base sequence shown in SEQ ID NO: 1-SEQ ID NO: 32; or

(2) An extended sequence having at least 40% sequence identity with any one of sequences shown in SEQ ID NO: 1-SEQ ID NO: 32.

It is understood that the sequence identity between the extended sequence and the base sequence may also be over 50%, 60%, 70%, 80%, or 90%. The targeting domain has a domain which is reversely complementary (partially complementary or fully complementary) to the targeting sequence (located on the SMAD binding site region or exon 2 region), and the extended sequence is a sequence that deletes, adds or substitute some bases based on the base sequence, but still has the basic sequence targeting function, such as a difference of no more than 10, 5, 3, or 1 nucleotide from the base sequence.

Further, in one embodiment, the targeting domain is selected from:

(1) Any of the base sequences shown in SEQ ID NO: 1-SEQ ID NO: 32; or

(2) An extended sequence having at least 90% sequence identity with any of the sequences shown in SEQ ID NO: 1-SEQ ID NO: 32.

The above sequences SEQ ID NO: 1-SEQ ID NO: 32 are shown in the following table:

TABLE 1
List of gRNA targeting domain sequence

| | gRNA targeting domain |
|---|---|
| number | sequence |
| SEQ ID NO: 1 | GUGCCAGCUUUUUCAGA |
| SEQ ID NO: 2 | UGUGCCAGCUUUUUCAGA |
| SEQ ID NO: 3 | GUGUGCCAGCUUUUUCAGA |
| SEQ ID NO: 4 | AGUGUGCCAGCUUUUUCAGA |
| SEQ ID NO: 5 | GAGUGUGCCAGCUUUUUCAGA |
| SEQ ID NO: 6 | GGAGUGUGCCAGCUUUUUCAGA |
| SEQ ID NO: 7 | UGGAGUGUGCCAGCUUUUUCAGA |
| SEQ ID NO: 8 | CUGGAGUGUGCCAGCUUUUUCAGA |
| SEQ ID NO: 9 | CCAGCUUUUUCAGACGG |
| SEQ ID NO: 10 | GCCAGCUUUUUCAGACGG |
| SEQ ID NO: 11 | UGCCAGCUUUUUCAGACGG |
| SEQ ID NO: 12 | GUGCCAGCUUUUUCAGACGG |
| SEQ ID NO: 13 | UGUGCCAGCUUUUUCAGACGG |
| SEQ ID NO: 14 | GUGUGCCAGCUUUUUCAGACGG |
| SEQ ID NO: 15 | AGUGUGCCAGCUUUUUCAGACGG |
| SEQ ID NO: 16 | GAGUGUGCCAGCUUUUUCAGACGG |
| SEQ ID NO: 17 | GUCUGCGCCAAGCAGCU |
| SEQ ID NO: 18 | CGUCUGCGCCAAGCAGCU |
| SEQ ID NO: 19 | GCGUCUGCGCCAAGCAGCU |
| SEQ ID NO: 20 | CGCGUCUGCGCCAAGCAGCU |
| SEQ ID NO: 21 | CCGCGUCUGCGCCAAGCAGCU |
| SEQ ID NO: 22 | GCCGCGUCUGCGCCAAGCAGCU |
| SEQ ID NO: 23 | UGCCGCGUCUGCGCCAAGCAGCU |
| SEQ ID NO: 24 | CUGCCGCGUCUGCGCCAAGCAGCU |
| SEQ ID NO: 25 | CGUCUGCGCCAAGCAGC |
| SEQ ID NO: 26 | GCGUCUGCGCCAAGCAGC |
| SEQ ID NO: 27 | CGCGUCUGCGCCAAGCAGC |
| SEQ ID NO: 28 | CCGCGUCUGCGCCAAGCAGC |
| SEQ ID NO: 29 | GCCGCGUCUGCGCCAAGCAGC |
| SEQ ID NO: 30 | UGCCGCGUCUGCGCCAAGCAGC |
| SEQ ID NO: 31 | CUGCCGCGUCUGCGCCAAGCAGC |
| SEQ ID NO: 32 | GCUGCCGCGUCUGCGCCAAGCAGC |

In the above sequences, SEQ ID NO: 1-16 are designed for SMAD binding site, and SEQ ID NO: 17-32 are designed for exon 2.

In one embodiment, the base sequence is selected from any of the sequences shown in SEQ ID NO: 1, SEQ ID NO: 4, SEQ ID NO: 6, SEQ ID NO: 8, SEQ ID NO: 9, SEQ ID NO: 12, SEQ ID NO: 14, SEQ ID NO: 16, SEQ ID NO: 18, SEQ ID NO: 20, or SEQ ID NO: 31. The present inventor has confirmed through experiments that the use of the above sequences had a good editing effect.

In the second aspect, the present application also disclosed a gRNA expression vector for targeting and editing CTGF gene, comprising a nucleotide sequence encoding the gRNA described above.

The expression vector described above can express gRNA for targeting and editing CTGF gene. It is understood that those skilled in the art can refer to conventional techniques to construct the expression vector described above.

In one embodiment, the expression vector comprises one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or a combination thereof. Examples of pol III promoters include, but are not limited to, U6 and H1 promoters. Examples of pol II promoters include, but are not limited to, retrovirus Rous sarcoma virus (RSV) LTR promoter (optionally with an RSV enhancer), cytomegalovirus (CMV) promoter (optionally with a CMV enhancer), SV40 promoter, dihydrofolate reductase promoter, β-actin promoter, phosphoglycerate kinase (PGK) promoter and EF1α promoter.

In one embodiment, the expression vector is selected from: plasmid, lentiviral vector, adenoviral vector, adeno associated viral vector, and herpes simplex viral vector. Further, the expression vector is selected from an adeno associated viral vector.

In the third aspect, the present application also disclosed a CRISPR system for targeting and editing CTGF gene, comprising the gRNA described above.

When the CRISPR system that targets and edits CTGF gene contacts target cells, it can change the gene sequence of cells, thereby affecting the fibrosis process to achieve the control of fibrotic diseases.

In one embodiment, the gRNA is a single molecule gRNA.

In one embodiment, the gRNA is a bimolecule gRNA.

In one embodiment, the CRISPR system further comprises a Cas enzyme.

In one embodiment, the Cas enzyme is CRISPR-related nuclease molecules or fusion proteins thereof, consisting of Type II, V, and VI nucleases, wherein Type II nuclease is Cas9, Type V nuclease is Cas 12 and Type VI nuclease is Cas 13. The Cas9 includes but not limited to SpCas9, SaCas9, Nme2Cas9, Nme3Cas9, CjCas9, NmCas9, FnCas9, nCas9, and dCas9 molecules, and fusion proteins and mutants thereof. The Cas12 includes but not limited to Cas12a, Cas12b, Cas12c, Cas12d, Cas12e, Cas 12f, Cas12g, Cas12h, Cas12i, Cas12j, Cas12k, Cas121 and Cas12m molecules, and fusion proteins and mutants thereof

In one embodiment, the CRISPR system further comprises a Cas9 nuclease. The gRNA described above, combined with Cas enzyme such as Cas9 nuclease, has high editing efficiency in cells, which can significantly reduce the overexpression of CTGF gene.

In one embodiment, the CRISPR system further comprises an AsCpf1 nuclease. The gRNA described above, combined with Cas enzyme such as AsCpf1 nuclease, has high editing efficiency in cells, which can significantly reduce the overexpression of CTGF gene.

In the fourth aspect, the present application also disclosed a composition for targeting and editing CTGF gene, comprising: a gRNA system and a Cas enzyme system, the gRNA system may directly or indirectly comprise the gRNA described above, the Cas enzyme system may directly or indirectly comprise a Cas enzyme.

It is understood that, comprising gRNA directly refers to directly use chemically synthesized gRNA for preparation, and comprising gRNA indirectly refers to produce gRNA through conventional means such as genetically engineered transcription; similarly, comprising a Cas enzyme directly refers to use purified Cas protein for preparation, and comprising a Cas enzyme indirectly refers to produce Cas enzymes through genetic engineering method.

In one embodiment, the gRNA system is selected from: the gRNA described above, or a nucleic acid encoding the gRNA described above; and the Cas enzyme system is selected from: Cas nucleases, or nucleic acids encoding Cas nucleases.

In one embodiment, the Cas enzyme is CRISPR-related nuclease molecules or fusion proteins thereof, consisting of Type II, V, and VI nucleases, wherein Type II nuclease is Cas9, Type V nuclease is Cas 12 and Type VI nuclease is Cas 13. The Cas9 includes but not limited to SpCas9, SaCas9, Nme2Cas9, Nme3Cas9, CjCas9, NmCas9, FnCas9, nCas9, and dCas9 molecules, and fusion proteins and mutants thereof. The Cas12 includes but not limited to Cas12a, Cas12b, Cas12c, Cas12d, Cas12e, Cas 12f, Cas12g, Cas12h, Cas12i, Cas12j, Cas12k, Cas121 and Cas12m molecules, and fusion proteins and mutants thereof

In the fifth aspect, the present application also disclosed a liposome, comprising an active ingredient and a lipid component as a carrier, the active ingredient comprising the gRNA, the gRNA expression vector, the CRISPR system, or the composition described above.

In the sixth aspect, the present application also disclosed a use of the gRNA, the gRNA expression vector, the CRISPR system, or the composition described above in preparation of a medicament for treating fibrotic diseases.

In the seventh aspect, the present application also disclosed a method for treating fibrotic diseases, comprising administering the gRNA, the gRNA expression vector, the CRISPR system, or the composition described above to a patient.

In one embodiment, the method comprises administering a therapeutically effective amount of the gRNA, the gRNA expression vector, the CRISPR system, or the composition described above to a patient.

In the eighth aspect, the present application also disclosed the gRNA, gRNA expression vector, CRISPR system, or composition described above for use in treating fibrotic diseases.

It is understood that the gRNA of the present application is designed for CTGF gene, and can edit CTGF gene, thereby having therapeutic effects on fibrotic diseases affected or regulated by CTGF gene.

In one embodiment, the fibrotic disease is pulmonary fibrosis.

Further, the fibrotic disease is idiopathic pulmonary fibrosis.

In one embodiment, the drug is administered by inhalation. Through direct pulmonary administration by inhalation, the dose can be reduced and systemic adverse effects can be avoided.

In the ninth aspect, the present application also disclosed a method for treating fibrotic diseases, comprising administrating the liposome described above.

In one embodiment, the fibrotic disease is pulmonary fibrosis.

In one embodiment, he subject is administrated through inhalation.

In one embodiment, the method comprises administering a therapeutically effective amount of the liposome described above to a patient.

Compared with the prior art, the present application has the following beneficial effects:

A gRNA targeting human CTGF gene of the present application, which is designed for targeting the SMAD binding site region or exon 2 region of CTGF gene promoter, so that the gRNA can reduce the overexpression of human CTGF gene through CRISPR-Cas gene editing system. The gRNA that targeting CTGF gene provided in the present application can reduce the overexpression of human CTGF gene by CRISPR-Cas gene editing system.

### BRIEF DESCRIPTION OF THE DRAWINGS

FIG. 1 is an electrophoretogram related to the exemplary plasmids in Example 1. Wherein: from left to right, electrophoretograms represent groups of Exon2-sgRNA2, SMAD-sgRNA2, and SMAD-sgRNA6 respectively.

FIG. 2 is a sequencing peak diagram of the exemplary plasmids of groups Exon2-sgRNA2, SMAD-sgRNA2, SMAD-sgRNA6 in Example 1.

FIG. 3 is a PCR electrophoresis result of the exemplary transfected cells in Example 1. Wherein: E1: the product amplified with Exon2-specific primers after editing by Exon2-sgRNA2; E2: the product amplified with Exon2-specific primers after editing by NC sgRNA; E3: the product amplified with Exon2-specific primers after extracting WT genome; S1: the product amplified with SMAD binding site-specific primers after editing by SMAD-sgRNA2; S2: the product amplified with SMAD binding site-specific primers after editing by SMAD-sgRNA6; S3: the product amplified with SMAD binding site-specific primers after editing by NC sgRNA; S4: the product amplified with SMAD binding site-specific primers after extracting WT genome.

FIG. 4 shows the gene editing efficiency of each group in Example 1.

FIG. 5 shows the test result of the relative expression level of hCTGF mRNA in Example 2.

### DETAILED DESCRIPTION OF THE PREFERRED EMBODIMENT

In order to facilitate understanding of the present application, a more comprehensive description of the application will be described below with reference to the related drawings. Preferred examples of the present application are shown in the drawings. However, the present application may be implemented in many different forms and is not limited to the examples described herein. On the contrary, the purpose of examples these embodiments is to make the understanding of the disclosure of the present application more thorough and comprehensive.

Unless otherwise defined, all technical and scientific terms used herein have the same meaning as commonly understood by a person skilled in the art. The terms used herein in the description of the present application are for the purpose of describing particular examples only and are not intended to limit the present application. As used herein, the term “and/or” means that any and all combinations of one or more of the relating listed items are included.

### Definitions

The gRNA molecule described in the present application comprises a targeting domain complementary to CTGF gene sequence, and a fixed sequence domain (a backbone sequence). The gRNA molecules described in the present application may be chemically modified on any nucleotide.

The “Cas enzyme” described in the present application refers to CRISPR-related nucleases, including but not limited to CRISPR-related nuclease molecules or fusion proteins thereof, consisting of Type II, V, and VI nucleases. Wherein Type II nuclease is Cas9, Type V nuclease is Cas 12 and Type VI nuclease is Cas 13.

The “Cas9” described in the present application includes but not limited to SpCas9, SaCas9, Nme2Cas9, Nme3Cas9, CjCas9, NmCas9, FnCas9, nCas9, and dCas9 molecules, and fusion proteins and mutants thereof

The “Cas12” includes but not limited to Cas12a, Cas12b, Cas12c, Cas12d, Cas12e, Cas 12f, Cas12g, Cas12h, Cas12i, Cas12j, Cas12k, Cas121 and Cas12m molecules, and fusion proteins and mutants thereof. In one embodiment, Cas12 is AsCpf1.

The reagents and materials in the following embodiments are all commercially available sources unless otherwise specified; experimental methods are conventional experimental methods in the art unless otherwise specified.

### EXAMPLE 1

CTGF gene promoter elements or exon 2 was edited by CRISPR gene editing methods.

### 1. Preparation of Vectors

1) Determine the gRNA targeting domain (the same as the sequence of the target sequence):

According to the sequence adjacent to the SMAD binding site of the human CTGF gene promoter region and the exon 2 sequence, gRNAs with a targeting domain length of 17 nt-24 nt were designed. In addition, a gRNA (Responsive-sgRNAx) targeting the GTGTCAAGGGGTC (SEQ ID NO: 57) sequence of the TGFβ response element adjacent to the SMAD binding site was designed. Some of them are shown in Table 2.

TABLE 2
The designed gRNA targeting domains

| | SEQ ID NO of | |
|---|---|---|
| | targeting | Targeting domain |
| gRNA name | domain | sequences |
| SMAD- | SEQ ID NO: 1 | GUGCCAGCUUUUUCAGA |
| sgRNA1 | | |
| SMAD- | SEQ ID NO: 4 | AGUGUGCCAGCUUUUUCAGA |
| sgRNA2 | | |
| SMAD- | SEQ ID NO: 6 | GGAGUGUGCCAGCUUUUUCAGA |
| sgRNA3 | | |
| SMAD- | SEQ ID NO: 8 | CUGGAGUGUGCCAGCUUUUUC |
| sgRNA4 | | AGA |
| SMAD- | SEQ ID NO: 9 | CCAGCUUUUUCAGACGG |
| sgRNA5 | | |
| SMAD- | SEQ ID NO: 12 | GUGCCAGCUUUUUCAGACGG |
| sgRNA6 | | |
| SMAD- | SEQ ID NO: 14 | GUGUGCCAGCUUUUUCAGACGG |
| sgRNA7 | | |
| SMAD- | SEQ ID NO: 16 | GAGUGUGCCAGCUUUUUCAGA |
| sgRNA8 | | CGG |
| Exon2-sgRNA1 | SEQ ID NO: 18 | CGUCUGCGCCAAGCAGCU |
| Exon2-sgRNA2 | SEQ ID NO: 20 | CGCGUCUGCGCCAAGCAGCU |
| Exon2-sgRNA3 | SEQ ID NO: 31 | CUGCCGCGUCUGCGCCAAG |
| | | CAGC |
| SMAD- | SEQ ID NO: 9 | CCAGCUUUUUCAGACGG |
| sagRNAl | | |
| SMAD- | SEQ ID NO: 12 | GUGCCAGCUUUUUCAGACGG |
| sagRNA2 | | |
| SMAD- | SEQ ID NO: 14 | GUGUGCCAGCUUUUUCAGACGG |
| sagRNA3 | | |
| SMAD- | SEQ ID NO: 16 | GAGUGUGCCAGCUUUUUCAGA |
| sagRNA4 | | CGG |
| Responsive- | SEQ ID NO: 33 | AGGAAUGCUGAGUGUCA |
| sgRNA1 | | |
| Responsive- | SEQ ID NO: 34 | CGGAGGAAUGCUGAGUGUCA |
| sgRNA2 | | |
| Responsive- | SEQ ID NO: 35 | CAGACGGAGGAAUGCUGAGU |
| sgRNA3 | | GUCA |

Among the gRNAs designed above, SMAD-sgRNAx, Exon2-sgRNAx, and Responsive-sgRNAx (x is the serial number) correspond to the subsequent construction of SpCas9 plasmid, and SMAD-sagRNAx (x is the serial number) corresponds to the subsequent construction of SaCas9 plasmid.

Sense strands and antisense strands (cacc was added to the 5′-end of the sense strand, and if the first nucleotide at the 5′-end of the sense strand is not guanine G, caccg was added to the 5′-end of the sense strand; aaac was added to the 5′-end of the antisense strand, if the first nucleotide of the 5′-end of the sense strand is not guanine G, C was added to the 3′-end of the antisense strand) corresponding to the DNA sequences of the gRNA targeting domain were synthesized using conventional methods.

The sense strands and antisense strands corresponding to the DNA sequences of the gRNA targeting sequences were mixed (each pair of sense strands and antisense strands [F/R strands] were tested separately), incubated at 95° C. in a PCR machine for 5 min. Next, the mixture was immediately taken out and incubated on ice for 5 min, and then annealed to form double-stranded DNA with sticky ends.

2 μL of the annealed product was used and diluted 500-fold with deionized water.

### 2) T4 Ligation Reaction

The PX459 plasmid (containing SpCas9 and AmpR corresponding sequences) was digested using restriction endonuclease Bbs I, the enzyme digestion effect was detected by electrophoresis, and the PX459 vector enzyme digestion product was recovered. The linearized PX459 was recovered through gel cutting, and then ligated with the annealed double-stranded DNA. The reaction system is showing in the following table. The annealed product and the linearized backbone vector were incubated at 16° C. in a PCR machine for 1 hour to fulfill the ligation, and SpCas9 plasmid was obtained.

TABLE 3
Ligation reaction system

| | |
|---|---|
| Annealed product | 2 μL |
| (diluted 1000-fold) | |
| Linearized backbone vector | 5 ng |
| Solution I (Takara) | 3 μL |
| Deionized water | Up to 6 μL |

Using a similar method as described above, the pX601 plasmid (containing SaCas9 and AmpR corresponding sequences) was digested with restriction enzyme Bsa I, recovered and ligated to obtain SaCas9 plasmid.

For example, the plasmid sequences constructed by SMAD-sgRNA2 group and SMAD-sagRNA2 group are shown in SEQ ID NO: 36 and SEQ ID NO: 37, respectively.

### 2. Plasmid Transformation and Coating Ampicillin-resistant Solid Culture Plate

1) In the super clean bench, all products in T4 ligation reaction were added into a tube (50 μL) comprising E. coli DH5a competent cells rapidly. Then, the tube was incubated on ice for 30 min.

2) The competent cells were immersed and bathed in water at 42° C. for 90 sec, and then incubated on ice for 2 min.

3) In the super clean bench, 400 μL of antibiotic-free LB medium was added to the bacterial solution. Then, the bacterial solution was put into a bacterial shaker and recovered at 200 rpm and 37° C. for 1 h. During recovery, the biochemical incubator was turned on and LB agar plates containing appropriate amount of ampicillin were dried in it.

4) The bacterial solution was centrifuged at room temperature and 12,000 rpm for 1 min, then most of the supernatant was removed using a pipette, about 50 μL supernatant was left to resuspend the precipitate thoroughly.

5) The bacterial solution was dropped on the edge of the ampicillin-containing LB agar plates and then streaked using a pipette tip. The plates were placed upside down in a biochemical incubator and incubated for 16-18 h.

3. Positive clones were selected, and plasmids were extracted and sequenced after expansion culture.

1) In the super clean bench, 7 single colonies were selected and added into 50 μL ampicillin-containing LB medium using 1-10 μL pipette tips separately. Then, the solution was pipetted several times to mix the bacteria with LB medium.

2) 2 μL of the bacterial solution was added to the colony PCR reaction solution (shown in the table below), then mixed thoroughly. PCR reaction was performed after collecting the liquid at the bottom of the tube through instantaneous centrifugation. The remained bacterial solution was placed in a biochemical incubator for further culture. The primer sequence of PX459-R is GAGTGAAGCAGAACGTGGGG (SEQ ID NO: 41) and the primer sequence of pX601-R is GCTGGCA AGTGTAGCGGTCA (SEQ ID NO: 42).

TABLE 4
PCR reaction solution corresponding to SpCas9 plasmid strain

| | | |
|---|---|---|
| 2 × Accurate Taq | 10 | μL; |
| Master Mix (dye plus) | | |
| U6 Promoter-F (10 μM) | 0.25 | μL; |
| PX459-R (10 μM) | 0.25 | μL; |
| Deionized water | 7.5 | μL |

TABLE 5
PCR reaction solution corresponding to SaCas9 plasmid strain

| | | |
|---|---|---|
| 2 × Accurate Taq | 10 | μL; |
| Master Mix (dye plus) | | |
| U6 Promoter-F (10 μM) | 0.25 | μL; |
| pX601-R (10 μM) | 0.25 | μL; |
| Deionized water | 7.5 | μL |

3) After PCR, 2 μL of PCR product was mixed with 1 μL of 6× Loading Buffer, then loaded into the sample wells of the agarose gel to run agarose gel electrophoresis.

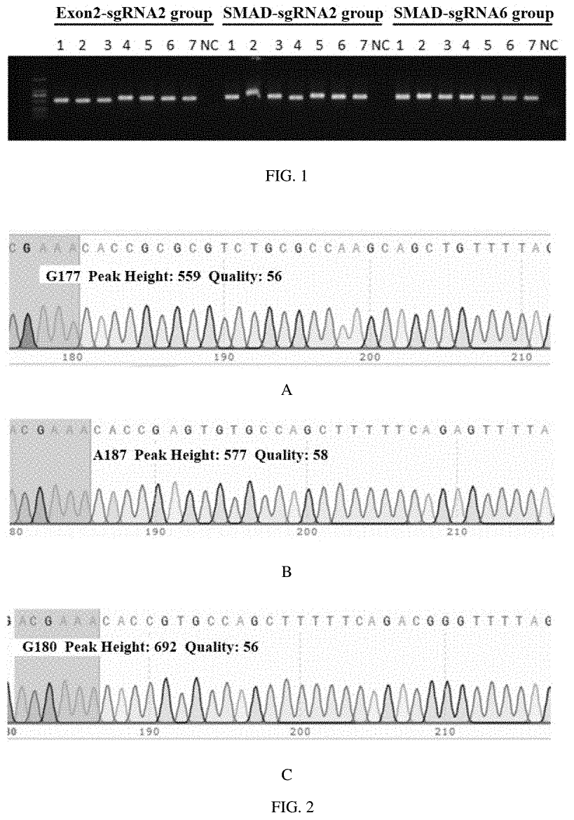

4) After finishing electrophoresis, the results were observed using the gel imaging system, and single clone in the electrophoresis strip with correct size and normal brightness were selected as positive clones. FIG. 1 showed an example of an electrophoretogram related to the PX459 plasmid, from left to right, respectively, electrophoretograms represent groups of Exon2-sgRNA2, SMAD-sgRNA2, and SMAD-sgRNA6, NC represents the negative control.

5) In super the clean bench, all the solutions of positive clones incubated in the biochemical incubator were added into 50 mL centrifuge tubes containing 5 mL ampicillin LB medium. After closing the lib, the centrifuge tubes were put into the bacterial shaker and tipsily fixed, cultured at 37° C. at 200 rpm for 16-18 hours.

6) The bacterial solution was used for plasmid extraction, and the operation procedure was performed according to the instructions of the plasmid DNA extraction kit, and eluted with 50 μL Elution Buffer finally.

7) According to the operation method of Qubit4 Fluorometer, the concentration of the plasmid was determined with the Qubit dsDNA BR Assay Kit.

8) One positive clone per plasmid was picked, 5-10 μL of plasmid was provided for Sanger sequencing, and the sequencing primer was the universal U6-Promoter-F (ACGATACAAGGCTGTTAGAG (SEQ ID NO: 43)). FIG. 2 showed an example of some sequencing results, wherein part A is a sequencing peak map for vector of Exon2-sgRNA2 group, part B is a sequencing peak map for vector of SMAD-sgRNA2 group, and part C is a sequencing peak map for vector of SMAD-sgRNA6 group.

### 4. Transfecting Cells, Extracting Genomes, and Identifying Genotypes

HEK293T cells were transfected with Lipofectamine2000 and the transfection reagent consisted of Lipofectamine2000 and Cas9 plasmid. Cells were seeded in 24-well plates with 5 x10⁵ cells per well and 500 ng plasmid were added.

After 72 h transfection, cells were collected and digested, then genome was extracted.

Sequences that approximate 500 bp from upstream to downstream of the gRNA binding site were amplified using the following specific primers

CTGF-SMAD-PCR-F:
(SEQ ID NO: 44)
CTCAGCGGGGAAGAGTTGTT
CTGF-SMAD-PCR-R:
(SEQ ID NO: 45)
TGCTGTTTGCCTCTTCAGCT
CTGF-EXON2-PCR-F:
(SEQ ID NO: 46)
CTCAGTCCGAGCGGTTTCTT
CTGF-EXON2-PCR-R:
(SEQ ID NO: 47)
ATGACCGCCGCCAGTATG
Responsive-PCR-F:
(SEQ ID NO: 48)
CTCTTTGGAGAGTTTCAAGAGCC
Responsive-PCR-R:
(SEQ ID NO: 49)
TCGAGCTGGAGGGTGGAGTC

Wherein, CTGF-SMAD-PCR-F and CTGF-SMAD-PCR-R were used to amplify fragments with approximate 500 bp from upstream to downstream of the SMAD binding site, and Responsive-PCR-F and Responsive-PCR-R were used to amplify fragments with approximate 250 bp from upstream to downstream of sequence GTGTCAAGGGGTC (SEQ ID NO: 57) of TGFβ response element, CTGF-EXON2-PCR-F and CTGF-EXON2-PCR-R were used to amplify fragments with approximate 500 bp from upstream to downstream of the cleavage site of exon 2 of CTGF gene.

The PCR reaction system was prepared as follows, with a total volume of 20 μL:

| | | |
|---|---|---|
| 2 × Accurate Taq | 10 | μL |
| Master Mix (dye plus) | | |
| Primer F (10 μM) | 0.25 | μL |
| Primer R (10 μM) | 0.25 | μL |
| Deionized water | 8.5 | μL |
| Genome DNA templete (50 ng) | 1 | μL |

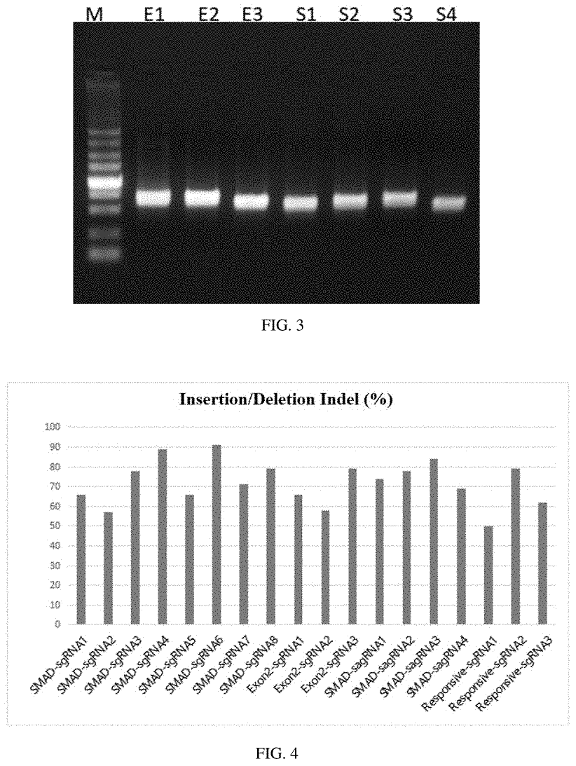

PCR products were detected by 1% agarose electrophoresis, and partial test results were shown in FIG. 3, wherein El is the product amplified with Exon2-specific primers after editing by Exon2-sgRNA2; E2 is the product amplified with Exon2-specific primers after editing by negative control sgRNA; E3 is the product amplified with Exon2-specific primers after extracting wild-type genome; S1 is the product amplified with SMAD binding site-specific primers after editing by SMAD-sgRNA2; S2 is the product amplified with SMAD binding site-specific primers after editing by SMAD-sgRNA6; S3 is the product amplified with SMAD binding site-specific primers after editing by negative control sgRNA; S4 is the product amplified with SMAD binding site-specific primers after extractingWT genome.

Then, the PCR products were recovered with kits and subjected to Sanger sequencing, and the experiments were repeated three times for each plasmid.

The sequencing results were imported into TIDE analysis website (https://ice.synthego.com/#/) to obtain gene editing efficiency, and the results were shown in the table below and FIG. 4.

TABLE 6
Gene editing efficiency of each group

| | Targeting | | Insertion/ |
|---|---|---|---|
| | domain | Cas | Deletion |
| gRNA name | sequences | enzymes | Indel(%) |
| SMAD-sgRNA1 | SEQ ID NO: 1 | SpCas9 | 66 |
| SMAD-sgRNA2 | SEQ ID NO: 4 | SpCas9 | 57 |
| SMAD-sgRNA3 | SEQ ID NO: 6 | SpCas9 | 78 |
| SMAD-sgRNA4 | SEQ ID NO: 8 | SpCas9 | 89 |
| SMAD-sgRNA5 | SEQ ID NO: 9 | SpCas9 | 66 |
| SMAD-sgRNA6 | SEQ ID NO: 12 | SpCas9 | 91 |
| SMAD-sgRNA7 | SEQ ID NO: 14 | SpCas9 | 71 |
| SMAD-sgRNA8 | SEQ ID NO: 16 | SpCas9 | 79 |
| Exon2-sgRNA1 | SEQ ID NO: 18 | SpCas9 | 66 |
| Exon2-sgRNA2 | SEQ ID NO: 20 | SpCas9 | 58 |
| Exon2-sgRNA3 | SEQ ID NO: 31 | SpCas9 | 79 |
| SMAD-sagRNA1 | SEQ ID NO: 9 | SaCas9 | 74 |
| SMAD-sagRNA2 | SEQ ID NO: 12 | SaCas9 | 78 |
| SMAD-sagRNA3 | SEQ ID NO: 14 | SaCas9 | 84 |
| SMAD-sagRNA4 | SEQ ID NO: 16 | SaCas9 | 69 |
| Responsive-sgRNA1 | SEQ ID NO: 33 | SpCas9 | 50 |
| Responsive-sgRNA2 | SEQ ID NO: 34 | SpCas9 | 79 |
| Responsive-sgRNA3 | SEQ ID NO: 35 | SpCas9 | 62 |

From the above results, it can be seen that the editing efficiency of all gRNA designed elaborately by present inventor can meet requirements, when targeting the SMAD binding site, sequence GTGTCAAGGGGTC (SEQ ID NO: 57) of TGFβ response element, and sequence GCTGCCGCGTCTGCGCCAAGCAGCT (SEQ ID NO: 58) of exon 2.

### EXAMPLE 2

Gene editing on pulmonary fibrosis cell models.

### 1. Experimental Materials

The Nucleic Acid Purification Kit and 2× Accurate Taq master Mix, Reverse Transcription Kit, and 2X SYBR® Green Pro Taq HS Premix were purchased from Accurate Biotechnology (Hunan) Co., Ltd., OPTI-MEM and Lipofectamine 3000 transfection reagents were purchased from Thermo company, and TGF-β1, SpCas9 protein, and SaCas9 protein were purchased from Novoprotein. The primers and related RNAs used in PCR and sequencing were synthesized by reagent companies. A549 cells are commercially available.

### 2. Experimental Methods

### 2.1 Experimental Grouping

The groups are as follows:

The A549 group: blank control without any treatment;

The A549 TGF-β1 group: cells were treated with TGF-β1 only, without transfecting with RNP.

SMAD-sgRNAx group (x is the serial number): wherein the gRNA targeting domain is the same as that of Example 1, the complete sequence of the gRNA consists of targeting domain-backbone sequence, the backbone sequence is GUUUUAGAGCUAGAAAUAGCAAGUUAAAAUAAGGCUAGUCCGUUAUCA ACUUGAAAAAGUGGCACCGAGUCGGUGCUUUU (SEQ ID NO: 50).

SMAD-sagRNAx group (x is the serial number): wherein the corresponding gRNA targeting domain is same as that of Example 1, the complete sequence of gRNA consists of targeting domain-backbone sequence, the backbone sequence consists of GUUUUAGUACUCUGGAAACAGAAUCUACUAAAACAAGGCAAAAUGCCG UGUUUAUCUCGUCAACUUGUUGGCGAGAUUUUU (SEQ ID NO: 51).

Responsive-sgRNAx group (x is the serial number): the gRNA in this group targets sequence GTGTCAAGGGGTC (SEQ ID NO: 57) of the TGFβ response element adjacent to the SMAD binding site, the gRNA targeting domain is shown in the following table, and the complete sequence of gRNA consist of targeting domain-backbone sequence, the backbone sequence is GUUUUAGAGCUAGAAAUAGCAAGUUAAAAUAAGGCUAGUCCGUUAUCA ACUUGAAAAAGUGGCACCGAGUCGGUGCUUUU (SEQ ID NO: 50).

The negative control group: the targeting sequence of gRNA is sequence TGTATGTCAGTGGACAGAAC at a distance of about 200 nt to the SMAD binding site of CTGF gene, and the complete sequence of gRNA is UGUAUGUCAGUGGACAGAACGUUUUAGAGCUAGAAAUAGCAAGUUAAA AUAAGGCUAGUCCGUUAUCAACUUGAAAAAGUGGCACCGAGUCGGUGC UUUU (SEQ ID NO: 52).

In fact, the gRNAs used in each group of SMAD-sgRNAx, SMAD-sagRNAx, and Responsive-sgRNAx are the same as the gRNA sequence transcribed by the plasmids in Example 1.

### 2.2 Experimental Steps

### 2.2.1. Cell Treatment

A549 cells were seeded into 24-well plates at 1×10⁵/well for one day before transfection.

### 2.2.2. RNP Transfection

### (1) RNP Transfection of SpCas9

The gRNA molecules in groups of SMAD-sgRNAx, Responsive-sgRNAx, and negative control were chemically synthesized.

Transfection complex A1 preparation: 6 pmol SpCas9 protein (Novoprotein, E365-01A) was added into 25 μL of OPTI-MEM medium (Thermo, 2120588), then 12 pmol gRNA was added to each group separately. The medium was mixed gently and placed at room temperature for 20 min to form RNP complex;

Transfection complex B1 preparation: 3 μL of Lipofectamine 3000 (Thermo, L3000-15) transfection reagent was added into 25 μL of OPTI-MEM medium. The medium was mixed gently and placed at room temperature for 5 min;

Transfection Complex Al was added into Transfection Complex B1 and gently mixed, then placed at room temperature for 15 min, followed by adding the mixture to the cells and culturing the cells for another 24 h.

### (2) RNP Transfection of SaCas9

The gRNA molecules inSMAD-sagRNAx group were chemically synthesized.

Transfection complex A2 preparation: 4 pmol SaCas9 protein (Novoprotein, E372-01A) and 8 pmol gRNA were added into μL of OPTI-MEM medium. The medium was mixed gently and placed at room temperature for 20 min to form RNP complex;

Transfection complex B2 preparation: 2 μL of Lipofectamine 3000 transfection reagent was added into 25 μL of OPTI-MEM medium. The medium was mixed gently and placed at room temperature for 5 min;

Transfection Complex A2 was added to Transfection Complex B2 and gently mixed, then placed at room temperature for 15 min, followed by adding the mixture to the cells and culturing the cells for another 24 h.

2.2.3. TGF-β1 (Novoprotein, P01137) was added to the cells to a final concentration of 10 ng/mL and the incubation was continued for 48 h.

2.2.4. Cell samples were collected to extract RNA using SteadyPure Universal RNA Extraction Kit (Accurate Biology, AG21017).

2.2.5. For RNA samples, gDNA was removed and reverse transcription reactions were performed using Evo M-MLV RT Kit with gDNA Clean for qPCR II (Accurate Biology, AG21017).

2.2.6. Relative quantitative QPCR was used to detect changes in the relative expression of CTGF mRNA with GAPDH as the reference gene. The primer design and QPCR reaction system are as follows:

Primer Design:

hCTGF-QPCR-F:
(SEQ ID NO: 53)
GCGTGTGCACCGCCAAAGAT
hCTGF-QPCR-R:
(SEQ ID NO: 54)
AACGTCCATGCTGCACAGGG
hGAPDH-QPCR-F:
(SEQ ID NO: 55)
GGAAACTGTGGCGTGATGGC
hGAPDH-QPCR-R:
(SEQ ID NO: 56)
GCTTCACCACCTTCTTGATGTC

QPCR reaction system:

| | |
|---|---|
| 2X SYBR ® Green | 10 μL |
| Pro Taq HS Premix | |
| Primer F (10 μM) | 0.4 μL |
| Primer R (10 μM) | 0.4 μL |
| cDNA Template | 2 μL |
| RNase free water | up to 10 μL |

### 2.2.7. Each Group Repeated the Experiment for 3 Times.

### 3. Experimental Results

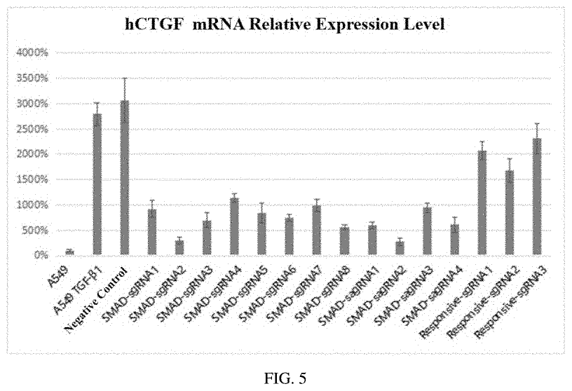

The results were analysed using the 2-ΔΔCT method, and the results were shown in the table below and FIG. 5.

TABLE 7
hCTGF mRNA relative expression

| | hCTGF | | hCTGF |
|---|---|---|---|
| | mRNA | | mRNA |
| | relative | | relative |
| groups | expression | group | expression |
| A549 | 100% | Negative | 3062% |
| | | control | |
| A549 TGF-β1 | 2794% | SMAD-sgRNA1 | 922% * |
| SMAD-sagRNA1 | 598% * | SMAD-sgRNA2 | 296% * |
| SMAD-sagRNA2 | 276% * | SMAD-sgRNA3 | 697% * |
| SMAD-sagRNA3 | 945% * | SMAD-sgRNA4 | 1139% * |
| SMAD-sagRNA4 | 608% * | SMAD-sgRNA5 | 840% * |
| Responsive-sgRNA1 | 2066% | SMAD-sgRNA6 | 753% * |
| Responsive-sgRNA2 | 1689% | SMAD-sgRNA7 | 992% * |
| Responsive-sgRNA3 | 2313% | SMAD-sgRNA8 | 565% * |

Note:
* indicates statistically significant difference (P < 0.05) compared to the Responsive-sgRNAx group.

The above experimental results proved that the modeling is successful. The inventor was surprised to find that the SMAD-sagRNAx and SMAD-sgRNAx were more effective at reducing hCTGF mRNA expression than the Responsive-sgRNAx group.

The above experimental results showed that gRNA or CRISPR-Cas system that target and edit SMAD binding sites can reduce the overexpression of CTGF gene more effectively than targeting and editing sequence GTGTCAAGGGGTC (SEQ ID NO: 57) of TGFβ response elements.

In addition, edition of SMAD binding site by using SaCas9 and SpCas9 with the gRNA of the present application separately significantly reduced the expression of hCTGF mRNA, both of them had good effects, although SaCas9 and SpCas9 used different Cas enzymes and involved different PAM sequences.

The technical features of the example described above may be combined arbitrarily. To make the description concise, not all of possible combinations of the technical features in the above examples were described, however, as long as there is no contradiction in the combination of these technical features, such combinations should be considered to fall into the scope of the present description.

The examples described above only show several examples of the present application, and the description thereof is more specific and detailed, but it cannot be understood as a limitation to the scope of the patent application. It should be noted that for an ordinary person skilled in the art, without departing from the concept of the present application, certain variations and improvements may also be made, which should all fall into the protection scope of the present application. Therefore, the scope of protection of the present application shall be defined by the attached claims.

What is claimed is:

1. A gRNA, wherein the gRNA can guide a Cas enzyme to target SMAD binding site region of a CTGF gene promoter, or the gRNA can guide a Cas enzyme to target and cleave CTGF gene exon 2 region.

2. The gRNA according to claim 1, wherein sequence of SMAD binding site region is shown in SEQ ID NO: 38 or a reverse complementary sequence thereof, and sequence of the exon 2 region is shown in SEQ ID NO: 39 or a reverse complementary sequence thereof

3. The gRNA according to claim 2, wherein sequence of the SMAD binding site sequence is shown in SEQ ID NO: 40 or a reverse complementary sequence thereof

4. The gRNA according to claim 1, which comprises a targeting domain selected from the group consisting of:
1) a base sequence as shown in any one of SEQ ID NO: 1-SEQ ID NO: 32; or
2) an extended sequence having at least 40% sequence identity with any one of SEQ ID NO: 1-SEQ ID NO: 32.

5. The gRNA according to claim 4, wherein the base sequence is selected from any one of SEQ ID NO: 1, SEQ ID NO: 4, SEQ ID NO: 6, SEQ ID NO: 8, SEQ ID NO: 9, SEQ ID NO: 12, SEQ ID NO: 14, SEQ ID NO: 16, SEQ ID NO: 18, SEQ ID NO: 20 or SEQ ID NO: 31.

6. A gRNA expression vector for targeting and editing CTGF gene, comprising a nucleotide sequence encoding the gRNA according to claim 1.

7. A CRISPR system for targeting and editing CTGF gene, comprising the gRNA according to claim 1.

8. The CRISPR system for targeting and editing CTGF gene of claim 7, which comprises a Cas enzyme.

9. The CRISPR system for targeting and editing CTGF gene of claim 8, wherein the Cas enzyme is Cas9, Cas12 or Cas13; the Cas9 includes but not limited to SpCas9, SaCas9, Nme2Cas9, Nme3Cas9, CjCas9, NmCas9, FnCas9, nCas9, and dCas9 molecules, and fusion proteins and mutants thereof; the Cas12 includes but not limited to Cas12a, Cas12b, Cas12c, Cas12d, Cas12e, Cas 12f, Cas12g, Cas12h, Cas12i, Cas12j, Cas12k, Cas121 and Cas12m molecules, and fusion proteins and mutants thereof

10. The CRISPR system for targeting and editing CTGF gene of claim 9, wherein the Cas enzyme is Cas9.

11. A composition for targeting and editing CTGF gene, comprising: a gRNA system and a Cas enzyme system, the gRNA system directly or indirectly comprising the gRNA according to claim 1, and the Cas enzyme system directly or indirectly comprising a Cas enzyme.

12. A composition for targeting and editing a CTGF gene, comprising: a gRNA system and a Cas enzyme system, the gRNA system directly or indirectly comprising a gRNA, and the Cas enzyme system directly or indirectly comprising a Cas enzyme;
wherein the gRNA system is selected from the group consisting of: a gRNA, or a nucleotide encoding a gRNA; and the Cas enzyme system is selected from a Cas enzyme, or a nucleotide encoding a Cas enzyme;
the gRNA is as defined in claim 1.

13. The composition for targeting and editing a CTGF gene according to claim 12, wherein the Cas enzyme is Cas9, Cas12 or Cas13; the Cas9 includes but not limited to SpCas9, SaCas9, Nme2Cas9, Nme3Cas9, CjCas9, NmCas9, FnCas9, nCas9, and dCas9 molecules, and fusion proteins and mutants thereof; the Cas12 includes but not limited to Cas12a, Cas12b, Cas12c, Cas12d, Cas12e, Cas12f, Cas12g, Cas12h, Cas12i, Cas12j, Cas12k, Cas121 and Cas12m molecules, and fusion proteins and mutants thereof

14. The composition for targeting and editing a CTGF gene according to claim 13, the Cas enzyme is Cas9.

15. A liposome, comprising an active ingredient and a lipid component as a carrier, the active ingredient comprising a gRNA, a gRNA expression vector comprising a nucleotide sequence encoding a gRNA, a CRISPR system comprising a gRNA, or a composition comprising a gRNA system and a Cas enzyme system;
the gRNA is as defined in claim 1.

16. A method for treating fibrotic diseases, comprising administrating a gRNA, a gRNA expression vector comprising a nucleotide sequence encoding a gRNA, a CRISPR system comprising a gRNA or a composition comprising a gRNA system and a Cas enzyme system, to a subject in need thereof;
wherein the gRNA is as defined in claim 1.

17. The method according to claim 16, wherein the fibrotic disease is pulmonary fibrosis.

18. The method according to claim 17, wherein the medicament is administered by inhalation.

19. A method for treating fibrotic diseases, comprising administrating the liposome targeting CTGF gene of claim 15;
preferably, the fibrotic disease is pulmonary fibrosis;
more preferably, the subject is administrated through inhalation.


SEQUENCE LISTING (58 sequences)

SEQ ID NO: 1 - RNA, 17 nt, synthetic construct
  source 1..17: mol_type = other RNA
gtgccagctttttcaga

SEQ ID NO: 2 - RNA, 18 nt, synthetic construct
  source 1..18: mol_type = other RNA
tgtgccagctttttcaga

SEQ ID NO: 3 - RNA, 19 nt, synthetic construct
  source 1..19: mol_type = other RNA
gtgtgccagctttttcaga

SEQ ID NO: 4 - RNA, 20 nt, synthetic construct
  source 1..20: mol_type = other RNA
agtgtgccagctttttcaga

SEQ ID NO: 5 - RNA, 21 nt, synthetic construct
  source 1..21: mol_type = other RNA
gagtgtgccagctttttcaga

SEQ ID NO: 6 - RNA, 22 nt, synthetic construct
  source 1..22: mol_type = other RNA
ggagtgtgccagctttttcaga

SEQ ID NO: 7 - RNA, 23 nt, synthetic construct
  source 1..23: mol_type = other RNA
tggagtgtgccagctttttcaga

SEQ ID NO: 8 - RNA, 24 nt, synthetic construct
  source 1..24: mol_type = other RNA
ctggagtgtgccagctttttcaga

SEQ ID NO: 9 - RNA, 17 nt, synthetic construct
  source 1..17: mol_type = other RNA
ccagctttttcagacgg

SEQ ID NO: 10 - RNA, 18 nt, synthetic construct
  source 1..18: mol_type = other RNA
gccagctttttcagacgg

SEQ ID NO: 11 - RNA, 19 nt, synthetic construct
  source 1..19: mol_type = other RNA
tgccagctttttcagacgg

SEQ ID NO: 12 - RNA, 20 nt, synthetic construct
  source 1..20: mol_type = other RNA
gtgccagctttttcagacgg

SEQ ID NO: 13 - RNA, 21 nt, synthetic construct
  source 1..21: mol_type = other RNA
tgtgccagctttttcagacgg

SEQ ID NO: 14 - RNA, 22 nt, synthetic construct
  source 1..22: mol_type = other RNA
gtgtgccagctttttcagacgg

SEQ ID NO: 15 - RNA, 23 nt, synthetic construct
  source 1..23: mol_type = other RNA
agtgtgccagctttttcagacgg

SEQ ID NO: 16 - RNA, 24 nt, synthetic construct
  source 1..24: mol_type = other RNA
gagtgtgccagctttttcagacgg

SEQ ID NO: 17 - RNA, 17 nt, synthetic construct
  source 1..17: mol_type = other RNA
gtctgcgccaagcagct

SEQ ID NO: 18 - RNA, 18 nt, synthetic construct
  source 1..18: mol_type = other RNA
cgtctgcgccaagcagct

SEQ ID NO: 19 - RNA, 19 nt, synthetic construct
  source 1..19: mol_type = other RNA
gcgtctgcgccaagcagct

SEQ ID NO: 20 - RNA, 20 nt, synthetic construct
  source 1..20: mol_type = other RNA
cgcgtctgcgccaagcagct

SEQ ID NO: 21 - RNA, 21 nt, synthetic construct
  source 1..21: mol_type = other RNA
ccgcgtctgcgccaagcagct

SEQ ID NO: 22 - RNA, 22 nt, synthetic construct
  source 1..22: mol_type = other RNA
gccgcgtctgcgccaagcagct

SEQ ID NO: 23 - RNA, 23 nt, synthetic construct
  source 1..23: mol_type = other RNA
tgccgcgtctgcgccaagcagct

SEQ ID NO: 24 - RNA, 24 nt, synthetic construct
  source 1..24: mol_type = other RNA
ctgccgcgtctgcgccaagcagct

SEQ ID NO: 25 - RNA, 17 nt, synthetic construct
  source 1..17: mol_type = other RNA
cgtctgcgccaagcagc

SEQ ID NO: 26 - RNA, 18 nt, synthetic construct
  source 1..18: mol_type = other RNA
gcgtctgcgccaagcagc

SEQ ID NO: 27 - RNA, 19 nt, synthetic construct
  source 1..19: mol_type = other RNA
cgcgtctgcgccaagcagc

SEQ ID NO: 28 - RNA, 20 nt, synthetic construct
  source 1..20: mol_type = other RNA
ccgcgtctgcgccaagcagc

SEQ ID NO: 29 - RNA, 21 nt, synthetic construct
  source 1..21: mol_type = other RNA
gccgcgtctgcgccaagcagc

SEQ ID NO: 30 - RNA, 22 nt, synthetic construct
  source 1..22: mol_type = other RNA
tgccgcgtctgcgccaagcagc

SEQ ID NO: 31 - RNA, 23 nt, synthetic construct
  source 1..23: mol_type = other RNA
ctgccgcgtctgcgccaagcagc

SEQ ID NO: 32 - RNA, 24 nt, synthetic construct
  source 1..24: mol_type = other RNA
gctgccgcgtctgcgccaagcagc

SEQ ID NO: 33 - RNA, 17 nt, synthetic construct
  source 1..17: mol_type = other RNA
aggaatgctgagtgtca

SEQ ID NO: 34 - RNA, 20 nt, synthetic construct
  source 1..20: mol_type = other RNA
cggaggaatgctgagtgtca

SEQ ID NO: 35 - RNA, 24 nt, synthetic construct
  source 1..24: mol_type = other RNA
cagacggaggaatgctgagtgtca

SEQ ID NO: 36 - DNA, 9177 bp, synthetic construct
  source 1..9177: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtgtgccagctttttcagagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 7446 bp, synthetic construct
  source 1..7446: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgccagctttttcagacgggttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 28 bp, Homo sapiens
  source 1..28: mol_type = other DNA
ccagctttttcagacggaggaatgctga

SEQ ID NO: 39 - DNA, 20 bp, Homo sapiens
  source 1..20: mol_type = other DNA
cgcgtctgcgccaagcagct

SEQ ID NO: 40 - DNA, 18 bp, Homo sapiens
  source 1..18: mol_type = other DNA
tttttcagacggaggaat

SEQ ID NO: 41 - DNA, 20 bp, synthetic construct
  source 1..20: mol_type = other DNA
gagtgaagcagaacgtgggg

SEQ ID NO: 42 - DNA, 20 bp, synthetic construct
  source 1..20: mol_type = other DNA
gctggcaagtgtagcggtca

SEQ ID NO: 43 - DNA, 20 bp, synthetic construct
  source 1..20: mol_type = other DNA
acgatacaaggctgttagag

SEQ ID NO: 44 - DNA, 20 bp, synthetic construct
  source 1..20: mol_type = other DNA
ctcagcggggaagagttgtt

SEQ ID NO: 45 - DNA, 20 bp, synthetic construct
  source 1..20: mol_type = other DNA
tgctgtttgcctcttcagct

SEQ ID NO: 46 - DNA, 20 bp, synthetic construct
  source 1..20: mol_type = other DNA
ctcagtccgagcggtttctt

SEQ ID NO: 47 - DNA, 18 bp, synthetic construct
  source 1..18: mol_type = other DNA
atgaccgccgccagtatg

SEQ ID NO: 48 - DNA, 23 bp, synthetic construct
  source 1..23: mol_type = other DNA
ctctttggagagtttcaagagcc

SEQ ID NO: 49 - DNA, 20 bp, synthetic construct
  source 1..20: mol_type = other DNA
tcgagctggagggtggagtc

SEQ ID NO: 50 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 51 - RNA, 81 nt, synthetic construct
  source 1..81: mol_type = other RNA
gttttagtactctggaaacagaatctactaaaacaaggcaaaatgccgtgtttatctcgtcaacttgttggcgagattttt

SEQ ID NO: 52 - RNA, 100 nt, synthetic construct
  misc_feature 1..100: note = gRNA Negative Control
  source 1..100: mol_type = other RNA
tgtatgtcagtggacagaacgttttagagctagaaatagcaagttaaaataaggctagtccgttatcaacttgaaaaagtggcaccgagtcggtgctttt

SEQ ID NO: 53 - DNA, 20 bp, synthetic construct
  source 1..20: mol_type = other DNA
gcgtgtgcaccgccaaagat

SEQ ID NO: 54 - DNA, 20 bp, synthetic construct
  source 1..20: mol_type = other DNA
aacgtccatgctgcacaggg

SEQ ID NO: 55 - DNA, 20 bp, synthetic construct
  source 1..20: mol_type = other DNA
ggaaactgtggcgtgatggc

SEQ ID NO: 56 - DNA, 22 bp, synthetic construct
  source 1..22: mol_type = other DNA
gcttcaccaccttcttgatgtc

SEQ ID NO: 57 - DNA, 13 bp, synthetic construct
  source 1..13: mol_type = other DNA
gtgtcaaggggtc

SEQ ID NO: 58 - DNA, 25 bp, synthetic construct
  source 1..25: mol_type = other DNA
gctgccgcgtctgcgccaagcagct